(19) United States
(12) Patent Application Publication
(10) Pub. No.: US 2007/0174088 A1
(43) Pub. Date: Jul. 26, 2007

(54) SYSTEM AND METHOD FOR PRODUCT LOCATION DETERMINATION AND INDICATION

(76) Inventors: Charles C. Koo, Palo Alto, CA (US); Dennis Wu, Palo Alto, CA (US); Roger Bertman, Los Altos, CA (US)

Correspondence Address: SQUIRE, SANDERS & DEMPSEY L.L.P, PATENT DEPARTMENT, ONE MARITIME PLAZA, SUITE 300, SAN FRANCISCO, CA, 94111-3492, US

(21) Appl. No.: 11/626,362

(22) Filed: Jan. 23, 2007

Related U.S. Application Data
(60) Provisional application No. 60/762,792, filed on Jan. 26, 2006.

(51) Int. Cl. G06Q 10/00; G06Q 50/00

(52) U.S. Cl. 705/2; 705/28

(57) ABSTRACT

A system and method recommend products (e.g., healthcare products) based on a user's requirements (e.g., symptoms). The system and method then indicate where the recommended product(s) is/are located.

### PRIORITY REFERENCE TO PRIOR APPLICATIONS

This application claims benefit of and incorporates by reference U.S. Patent Application No. 60/762,792, filed on Jan. 26, 2006, by inventors Charles C. Koo et al. This application also incorporates by reference U.S. patent application Ser. No. 11/315,410 filed on Dec. 22, 2005.

### TECHNICAL FIELD

This invention relates generally to healthcare product recommendation, and more particularly, but not exclusively, provides a system and method for recommending a healthcare product and indicating the recommended product's location.

### BACKGROUND

A retail store or a pharmacy has tens of thousands of products on the shelf, many of which are complex and require advice from experts. In some cases, consumers do not know the product but only know the problem that they want to solve. For example, there are more than two thousand over-the-counter (OTC) drug products for the treatment of a variety of symptoms and conditions in a typical store. Consumers entering the store to find the right product to treat their particular symptoms and conditions are faced with a confusing set of choices, and they are ill-equipped and uncertain of how to make the right choice to meet their needs. Faced with this uncertainty, they typically spend a good deal of time, up to 10 to 20 minutes, picking up packages and then comparing package descriptions trying to understand the ingredients of each product and how those ingredients relate to the particular symptoms they want to relieve, diseases or conditions they have, or other considerations such as age or allergies. Their intent is to find the right product which has all of the ingredients they need with no ingredients that they don't need or their conditions prohibit. Frequently, after searching on their own, in vain consumers ask a pharmacist for advice. Similar problem exists in most health-and-beauty products such as vitamins, supplements and cosmetics (including skin care products).

As such, a new system and method is needed for recommending products and indicating their location.

### SUMMARY

A location database associates each product, uniquely identified by its Universal Product Code (UPC), with coordinates of the product on the specific shelf. It is based on the retailer's mechanized “plan-o-gram” which specifies the aisle and shelf location of each product in each store, based on the Universal Product Code (UPC) for each product. The system matches the UPC code for the product selected by the consumer to product locations from the specific store plan-o-gram and then displays the location using an innovative aisle/shelf map display.

In an embodiment a method comprises: receiving a symptom; determining one or more healthcare products based on the symptom; determining a location of at least one of the one or more determined products; and indicating the location of at least one of the one or more determined products.

In an embodiment, a system comprises: a recommendation engine, a location engine and a user interface. The recommendation engine determines one or more healthcare products based on a symptom. The location engine, which is communicatively coupled to the recommendation engine, determines a location of at least one of the one or more determined products. The user interface, which is communicatively coupled to the location engine, indicates the location of the at least one of the one or more determined products.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a diagram illustrating a kiosk;

FIG. 2 is a block diagram illustrating components of the kiosk of FIG. 1;

FIG. 3 is a block diagram illustrating a persistent memory of the kiosk;

FIG. 4 is a diagram illustrating an example of a location database of the persistent memory;

FIG. 5 is a diagram illustrating an example display of a product location indication; and

FIG. 6 is a flowchart illustrating a method of recommending a product and indicating its location.

### DETAILED DESCRIPTION OF THE ILLUSTRATED EMBODIMENTS

The following description is provided to enable any person having ordinary skill in the art to make and use the invention and is provided in the context of a particular application and its requirements. Various modifications to the embodiments will be readily apparent to those skilled in the art, and the principles defined herein may be applied to other embodiments and applications without departing from the spirit and scope of the invention. Thus, the present invention is not intended to be limited to the embodiments shown, but is to be accorded the widest scope consistent with the principles, features and teachings disclosed herein.

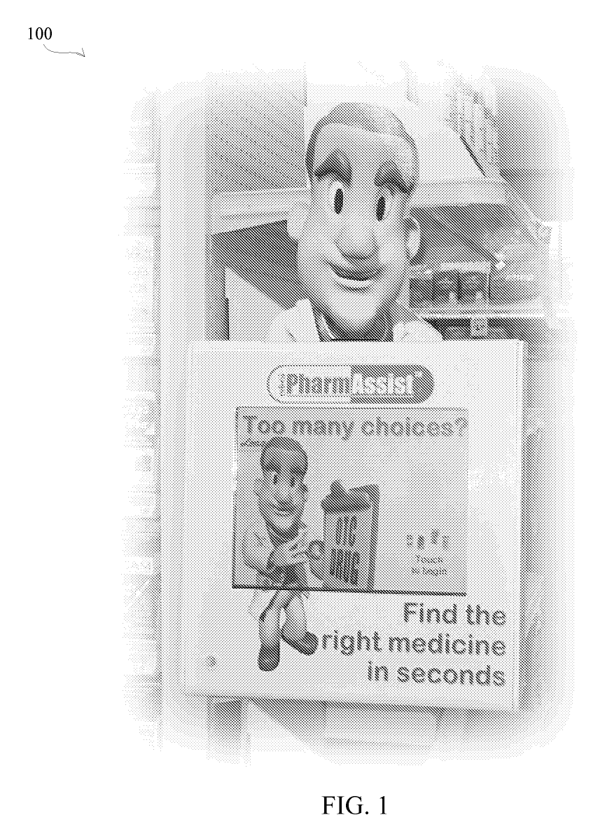

FIG. 1 is a diagram illustrating a kiosk 100. The kiosk 100 implements a process which recommends the correct products to an individual consumer within a retailing/pharmacy environment for the problem (e.g., relieve a set of symptoms and conditions) specified by the consumer. In addition, the kiosk 100 determines the location(s) of the recommended product(s) and graphically indicates determined location to the consumer.

The kiosk 100 uses search algorithms, e.g., deductive search algorithms, to identify all healthcare products (e.g., OTC drug products) that, in one embodiment, contain the minimum active ingredients to address the symptoms and conditions specified by a consumer. The deductive search algorithms dynamically narrow the universe of potential results as the user specifies symptoms and conditions. Interactions between the search algorithms and a knowledge base recognize symptoms and conditions that are causal or mutually exclusive. As the set of symptoms is specified, other symptoms or conditions that can not coexist in a diagnosis or recommendation are eliminated. Similarly, in one embodiment, as conditions are specified which preclude the use of certain active ingredients, those active ingredients are removed from the list of potential remedies. These algorithms result in a dynamic, real-time identification of possible diseases and treatments. Once the minimal list of active ingredients has been determined through this search algorithm, that list is used to determine all healthcare products that contain those active ingredients. This result meets the criterion of treating all specified symptoms within the constraints of the specified conditions. (In an embodiment of the invention, the minimal active ingredients need not be considered in product selection. In an embodiment, the minimal ingredients rule can be relaxed under certain business considerations.)

The final step is to “filter” out all healthcare/personal care products that contain any active ingredients other than those in the minimal list. This step assures that the consumer is recommended only the products which contain only the precise ingredients required to treat the specified symptoms recognizing the specified condition constraints to minimize the potential side effect from unnecessary ingredients.

Once the set of recommended healthcare products that, in one embodiment, have the minimal set of ingredients and meeting all condition constraints has been determined, the kiosk 100 lists the recommended products. In addition, the kiosk 100 then determines where the recommended products are located and graphically indicates their location.

In an embodiment of the invention, in addition to or in place of the kiosk 100, a web server can communicate with users coupled to a network and provide the functionality of the kiosk 100 without the need for a user to be physically in a store. For example, the functionality of the kiosk can be incorporated in an online healthcare product store. The kiosk 100 will be discussed in further detail below.

The kiosk 100 is designed to meet a number of criteria which enable it to effectively engage and interact with consumers and to operate efficiently in a retail store environment. These design criteria include the following:
Requires no floor space (“zero footprint”);
Occupies minimal shelf space;
Is fully self-contained (i.e. includes CPU, touch screen, power supply, speaker system, motion detectors, etc., and requires no integration with retailer IT systems);
“Plug and play” installation;
Remote management and support;

In addition to meeting these criteria, the kiosk also has a “Trade Dress” which provides an attractive, engaging presence for the consumer.

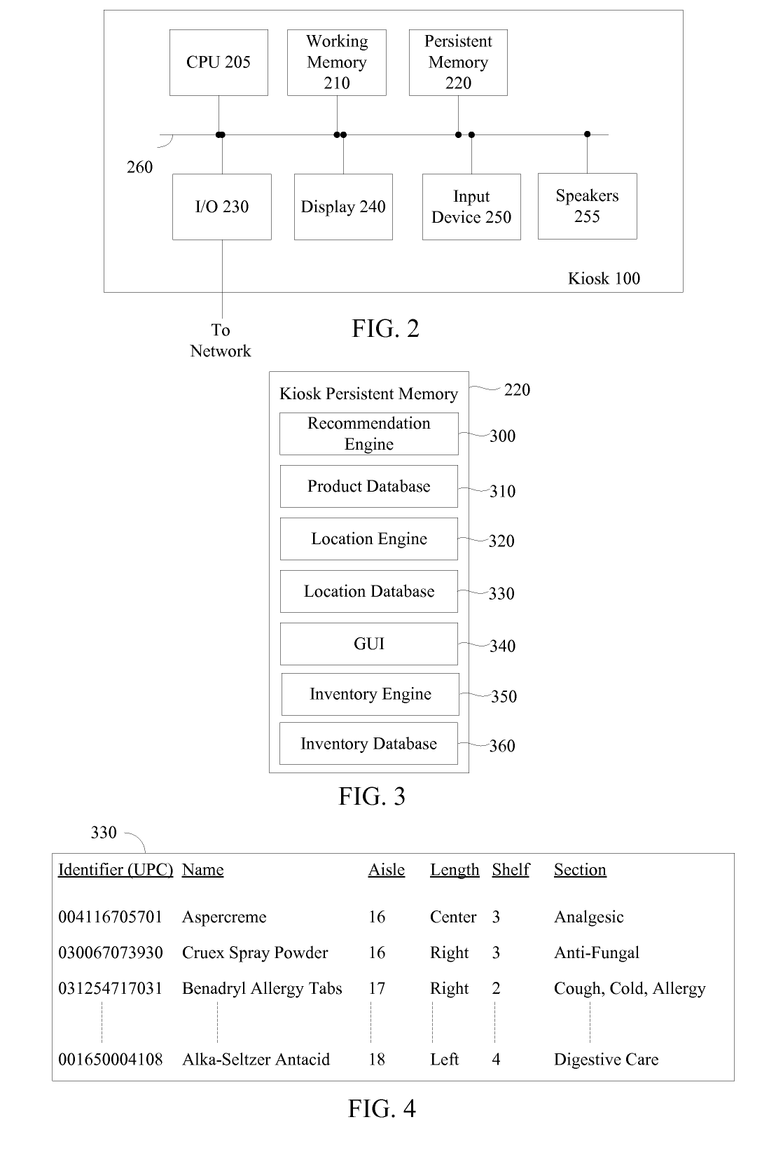

FIG. 2 is a block diagram illustrating components of the kiosk 100. The kiosk 100 includes a central processing unit (CPU) 205; working memory 210; persistent memory 220; input/output (I/O) interface 230; display 240; input device 250; and speakers 255 all communicatively coupled to each other via a bus 260. The CPU 205 may include an INTEL PENTIUM microprocessor, a Motorola POWERPC microprocessor, or any other processor capable to execute software stored in the persistent memory 220. The working memory 210 may include random access memory (RAM) or any other type of read/write memory devices or combination of memory devices. The persistent memory 220 may include a hard drive, read only memory (ROM) or any other type of memory device or combination of memory devices that can retain data after the kiosk 100 is shut off. In an embodiment, the I/O interface 230 is communicatively coupled, via wired or wireless techniques, to a network, such as the Internet. The display 240 may include a flat panel display, cathode ray tube display, or any other display device. The input device 250, may include a keyboard, mouse, touch screen or other device for inputting data, or a combination of devices for inputting data. The speakers 255, which are optional like other components of the invention, emit sound in mono or stereo.

In an embodiment of the invention, the kiosk 100 may also include additional devices, such as network connections, additional memory, additional processors, LANs, input/output lines for transferring information across a hardware channel, the Internet or an intranet, etc. In an embodiment of the invention, the kiosk 100 includes a motion sensor coupled to the bus 260 that activates the display 240 and speakers 255, if any. One skilled in the art will also recognize that the programs and data may be received by and stored in the kiosk 100 in alternative ways.

FIG. 3 is a block diagram illustrating a persistent memory 220 of the kiosk 100. The persistent memory 220 includes a recommendation engine 300, a product database 310, a location engine 320, a location database 330, a graphical user interface (GUI) 340, an inventory engine 350 and an inventory database 360. In an embodiment of the invention, the product database 310 and the location database 330 are combined into a single database. The recommendation engine 300 recommends products based on symptoms entered. In an embodiment of the invention, products include healthcare products, beauty products, or any other product that a pharmacy and/or store might sell. The recommendation engine 300, in an embodiment of the invention, uses the method disclosed in U.S. patent application Ser. No. 11/315,410 filed on Dec. 22, 2005, e.g., an ontological searching method.

The product database 310 includes products that are available for sale, for example, healthcare products. The recommendation engine 300 recommends products listed in the database 310. Each product in the database 310 can have an associated symptom(s) and/or disease(s) for which the recommendation engine 300 searches for. For example, if a consumer specifies the symptom “sneezing,” the recommendation engine 300 can search for this symptom in the database 310 and return products that deal this symptom. Alternatively or in addition, the recommendation engine 300 can infer that the consumer is suffering from a cold or allergies (based on other additional symptoms entered) and then search for products for colds and/or allergies. The database 310 can also include further product information such as images of the product, ingredients, size, price, equivalent products, etc.

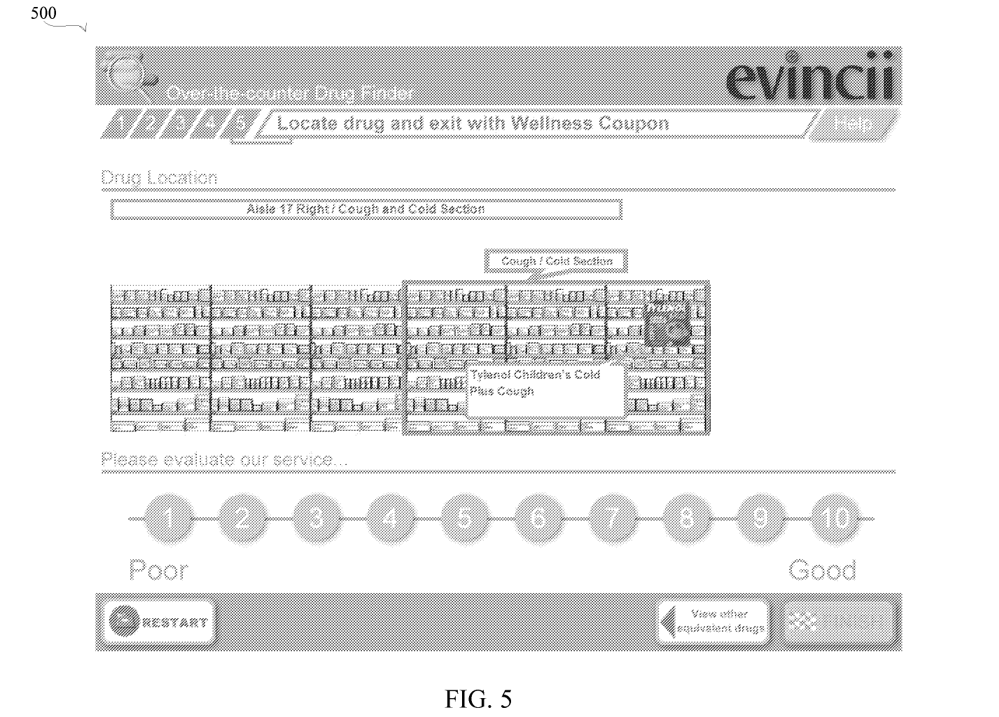

Once a product is selected by a user, the location engine 320 determines the specific location of the selected product on the shelf by looking up the product in the location database 330 by an identifier, such as a UPC. The location includes an aisle identifier in the store, a particular shelf, distance from one end of the aisle and/or section (e.g., cold section, digestive care section, etc.). The GUI 340 then displays the determined location. An example display is shown in FIG. 5, as will be discussed further below.

The location database 330 includes an identifier, product name, aisle identifier, length (location along the aisle), shelf number (e.g., height), and/or section. The location database 330 can be populated manually by the store, use data imported from a store's plan-o-gram.

In an embodiment, the inventory engine 350 determines if a selected product is in inventory by looking up the selected product in the database 360, which lists inventory of products in a store. If the product is not in inventory, then GUI 340 can inform the user of this or modify the list of products determined by the recommendation engine 300 to only products in inventory.

FIG. 4 is a diagram illustrating an example of the location database 330. Each product sold in a store is listed in the database 330 along with its location. Each product is identified with an identifier, such as a UPC.

FIG. 5 is a diagram illustrating an example display 500 of a product location indication. The display 500 indicates which aisle and which section of the aisle the determined product is located. In addition, the display shows the height and product section wherein the product is located. In an embodiment, the GUI 340 shows the location of all recommended products simultaneously in a single display.

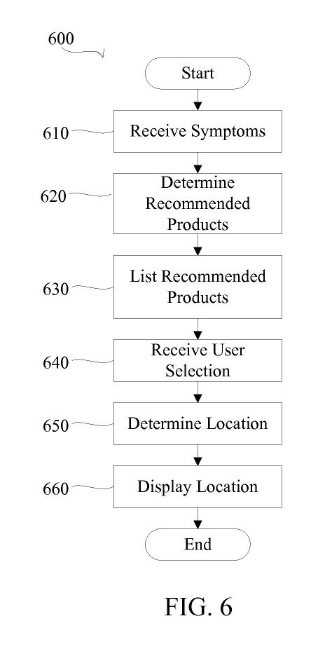

FIG. 6 is a flowchart illustrating a method 600 of recommending a product and indicating its location. First, symptoms are received (610), then recommended products are determined (620) based on the received symptoms. The recommended products are then displayed (630), e.g., in a list. A user then makes a selection, which is received (640). The location is then determined (650) and displayed (660). The method 600 then ends.

In an embodiment of the invention, the determining (620) only determines products in inventory by also checking an inventory database. In another embodiment, the displaying (630) also displays if the displayed products are in inventory by checking an inventory database.

The foregoing description of the illustrated embodiments of the present invention is by way of example only, and other variations and modifications of the above-described embodiments and methods are possible in light of the foregoing teaching. For example, embodiments of the invention can be used for any sort of product location determination and is not limited to healthcare products. Further, components of this invention may be implemented using a programmed general purpose digital computer, using application specific integrated circuits, or using a network of interconnected conventional components and circuits. Connections may be wired, wireless, modem, etc. The embodiments described herein are not intended to be exhaustive or limiting. The present invention is limited only by the following claims.

What is claimed is:

1. A method, comprising:
receiving a symptom or condition;
determining one or more healthcare products based on the symptom or the condition;
determining a location of at least one of the one or more determined products; and
indicating the location of the at least one of the one or more determined products.

2. The method of claim 1, further comprising receiving a user selection of one or more products and wherein the determining the location determines the location of a user-selected product.

3. The method of claim 1, wherein the indicating includes graphically indicating the location.

4. The method of claim 3, wherein the graphically indicating displays an aisle with the determined location of at least one or more determined products thereon.

5. The method of claim 4, wherein the graphically indicating displays a product section on the aisle.

6. The method of claim 1, wherein the indicating indicates an aisle and section wherein the at least one of the one or more products is located.

7. The method of claim 1, wherein the determining one or more healthcare products uses an ontological search.

8. The method of claim 1, wherein the determining a location uses data from a store's plan-o-gram.

9. The method of claim 1, further comprising determining if the one or more healthcare products are in inventory and wherein indicating further indicates if the product is in inventory.

10. The method of claim 1, further comprising determining if one or more healthcare products are in inventory and wherein the indicating only indicates locations of products in inventory.

11. A system, comprising:
a recommendation engine for determining one or more healthcare products based on a symptom;
a location engine, communicatively coupled to the recommendation engine, for determining a location of at least one of the one or more determined products; and
a user interface, communicatively coupled to the location engine, for indicating the location of the at least one of the one or more determined products.

12. The system of claim 11, wherein the user interface is further for receiving a user selection of the one or more products and wherein the location engine further determines the location of the user-selected product.

13. The system of claim 11, wherein the user interface graphically indicates the location.

14. The system of claim 13, wherein the user interface displays an aisle with the determined location of the least one or more determined products thereon.

15. The system of claim 14, wherein the user interface displays a product section on the aisle.

16. The system of claim 11, wherein the user interface indicates an aisle and section wherein the at least one of the one or more products is located.

17. The system of claim 11, wherein the recommendation engine uses an ontological search.

18. The system of claim 11, wherein the location engine uses data from a store's plan-o-gram.

19. The system of claim 11, further comprising an inventory engine for determining if the one or more healthcare products are in inventory and wherein the user interface further indicates if the product is in inventory.

20. The system of claim 11, further comprising an inventory engine for determining if the one or more healthcare are in inventory and wherein the user interface only indicates locations of products in inventory.

21. A computer-readable medium having stored thereon instructions to cause a computer to execute a method, the method comprising:
receiving a symptom;
determining one or more healthcare products based on the symptom;
determining a location of at least one of the one or more determined products; and
indicating the location of the at least one of the one or more determined products.

22. A system, comprising:
means for receiving a symptom or a problem description;
means for determining one or more healthcare products based on the symptom;
means for determining a location on a shelf of at least one of the one or more determined products; and
means for indicating the location of the at least one of the one or more determined products on the shelf.
